(19) United States
(12) Patent Application Publication
(10) Pub. No.: US 2007/0016301 A1
(43) Pub. Date: Jan. 18, 2007

(54) Multi-axial interbody spacer device

(75) Inventors: Jaime Martinez, Pompton Plains, NJ (US); Alexandre M. DiNello, Cotuit, MA (US)

Correspondence Address: GIBBONS, DEL DEO, DOLAN, GRIFFINGER & VECCHIONE, 1 RIVERFRONT PLAZA, NEWARK, NJ, 07102-5497, US

(73) Assignee: Medical Device Concepts LLC.

(21) Appl. No.: 11/181,050

(22) Filed: Jul. 14, 2005

(51) Int. Cl. A61F 2/44

(52) U.S. Cl. 623/17.13

(57) ABSTRACT

An interbody spacer for implantation into the intervertebral space between adjacent vertebrae of a patient's spinal column. The interbody spacer is a monolithic body having a predetermined slot design formed therein that provides flexibility to the spacer to allow movement of the adjacent vertebrae along multiple axies, that is, the patient can move both side to side and front to back and rotation. In one embodiment, the slot design is a spiral slot and in an alternate embodiment, the slot design comprises pairs of oppositely disposed, co-planar slots with alternating pairs of the slots offset about 90 degrees from the preceding pair of slots. Further embodiments include interbody spacers having the aforedescribed slot designs for implantation from the posterior, the anterior or translaterally into the intervertebral space while still allowing the multi-axial flexibility of the adjacent vertebrae.

### FIELD OF THE INVENTION

The present invention relates generally to prosthesis devices for insertion into the intervertebral space between adjacent vertebrae of a patient's spinal column to replace at least a portion of a disc normally occupying that intervertebral space and, more particularly, to a monolithic interbody spacer that allows relative motion between the adjacent vertebrae.

### BACKGROUND OF THE INVENTION

In the field of spinal devices there currently is a wide use of spinal fusion procedures that basically fuse adjacent spinal vertebrae together and such procedures are quite successful, provide adequate spacing of the disc space, and relieve the pain of the patient. As a downside, however, the spinal fusion technique does limit the range of motion of the spine and can lead to further degeneration of adjacent vertebrae.

As such, more recent techniques involve the use of artificial disc replacements and therefore provide more flexibility to the spine. There are a number of differing types of artificial disc replacements including mechanical devices, elastomeric disc devices and hybrids of the above. The general aim of the prosthetic disc or device is to provide a junction between adjacent vertebrae in replacement of the natural disc that may be partially or fully removed, and to thereby allow the same freedom of movement between the adjacent vertebrae that was allowed by the normal disc. The lack of movement between adjacent vertebrae, such as is the result of fusing of adjacent vertebrae together, has been recognized to result in more rapid deterioration of the further adjacent levels of vertebrae. That movement, therefore, should be multi-axial, that is, the movement of adjacent vertebrae should be controlled in bending and extension or side to side motion about a plurality of axes.

In addition, it would be preferred in such prosthetic devices that the device itself by monolithic having no moving parts or components that can fail mechanically and/or generate debris that could further exacerbate the difficulties of the patient and require further surgery. One monolithic prosthetic device that is insertable intermediate adjacent vertebra is shown and described in U.S. Pat. No. 6,136,031 of Middleton where the device has a longitudinal axis and a lateral axis transverse to the longitudinal axis and where the device is configured to have a slit with a longitudinal component of direction and a slit with a lateral component of direction to promote bone fusion and provide slight compression. While the Middleton device is monolithic, it would be advantageous for the prosthetic device to have a greater range of flexibility, particular along more than one axis to allow the freedom of movement of the patient at least along a side to side direction and a front to back direction.

There is, therefore a need for a monolithic prosthetic device that is implantable in the space intermediate adjacent vertebrae in order to provide support for such vertebrae and yet which allows a multi-axial movement between those vertebrae.

### SUMMARY OF THE INVENTION

The present invention relates to a monolithic multi axial interbody spacer device that can be implanted intermediate adjacent vertebrae of a patient's spinal column and which provides support for the vertebrae as well as providing flexibility therebetween.

As a desirable characteristic of the present replacement interbody spacer, the present device is capable of being constructed as a custom device, that is, the degree of flexibility can be controlled by the manufacturer as specified by the physician, so that the physician can assess the particular needs of each individual patient and surgically insert a device that has the exact flexibility required by the individual patient, for example, the degree of flexibility may be varied in accordance with the conditions of the disc of the patient and whether that disc has been fully or only partially removed.

The intervertebral prosthesis of the present invention includes a tubular body having a longitudinal axis and an outer peripheral surface and has an upper surface adapted to engage one of the adjacent vertebra and a lower surface that is adapted to engage the other of the adjacent vertebra. The tubular body is specially constructed to have a flexibility that can be determined by the designer of the device so as to provide the customized degree of flexibility that is desired by the physician in accordance with the particular needs of the patient.

As such, the flexibility of the interbody spacer can be directional, that is, the amount of movement along differing axies of movement can be designed to be similar to the normal biomechanical function of a healthy disc. Thus, the present interbody spacer can be designed and constructed to allow a front to back movement, i.e. flexion/extension in the range of from about greater than 0 to about 30 degrees of movement while, at the same time allowing lateral or side to side movement of the spinal column to range from about greater than 0 to about 20 degrees and rotational movement of the spinal column to be from about greater than 0 to about 15 degrees in rotation.

The tubular body has one or more slots formed therein and, in one embodiment the slot is one or more spiral, or helical slots that are formed in the outer periphery of the tubular body in the manner shown and described in U.S. Pat. No. 5,488,761 of Leone, the disclosure of which is incorporated herein in its entirety by reference. In another embodiment the slots are formed in oppositely disposed pairs of co-planar slots with each pair of oppositely disposed slots offset angularly with respect to the adjacent pair of slots as shown and described in copending U.S. patent application Ser. No. ______ filed Jun. 3, 2005 and entitled “FLEXIBLE SHAFT”, the disclosure of which is incorporated herein in its entirety by reference. As a further alternative, the body may be constructed having leaf springs incorporated thereinto.

In alternative embodiments, the use of the slotted tubular body can be employed for differing intervertebral prosthetic devices with different insertion techniques and inserted from different orientations. For example the use of the slotted tubular body constructed in accordance with the present invention can be inserted intermediate the adjacent vertebrae by an anterior technique, i.e. Anterior Lumbar Interbody Fusion/Motion (ALIF); by a posterior technique, Posterior Lumbar Interbody Fusion/Motion (PLIF) or even by a lateral insertion technique, (Translateral Lumbar Interbody Fusion/Motion). In any of the above techniques, the main body is unique in that it is slotted in accordance with the present invention so that there is a monolithic body allowing multi-axial movement between the adjacent vertebrae.

These and other features and advantages of the present invention will become more readily apparent during the following detailed description taken in conjunction with the drawings herein.

### BRIEF DESCRIPTION OF THE DRAWINGS

FIG. 1 is a horizontal cross-section view of a spinal column;

FIG. 2 is perspective view of an exemplary interbody spacer constructed in accordance with the present invention;

FIG. 3 is perspective view of another embodiment of the interbody spacer of the present invention;

FIG. 4 is a front view of a spinal column of a patient having an interbody spacer implanted therein;

FIG. 5 is a side view illustrating an exemplary implantation technique for an interbody spacer into the spinal column of a patient;

FIG. 6 is a further embodiment of the interbody spacer of the present invention;

FIG. 7 is a still further embodiment of the interbody spacer of the present invention;

FIG. 8 is a side view of a spinal column illustrating the front to back movement of the spinal column;

FIG. 9 is a side view of the spinal column of FIG. 8 illustrating the side to side movement of the spinal column; and

FIG. 10 is a top view of a vertebra illustrating the rotational movement between adjacent vertebrae of the spinal column.

### DETAILED DESCRIPTION OF THE INVENTION

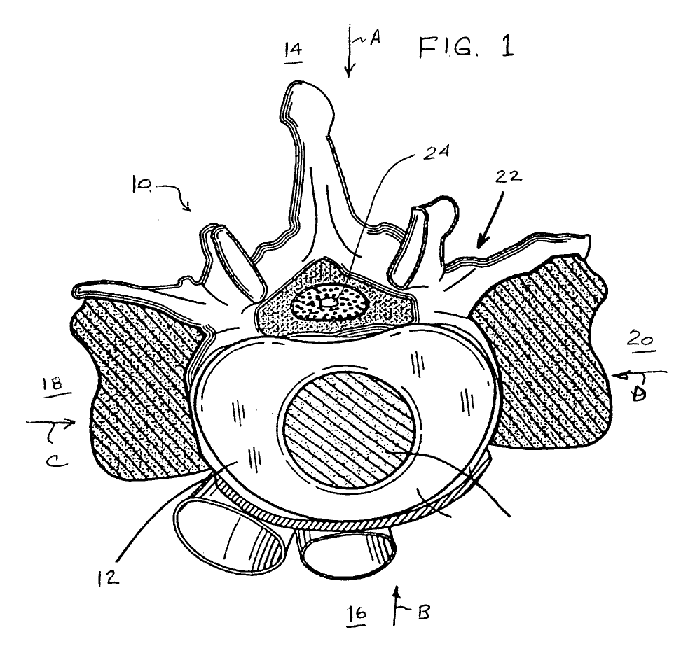

Referring now to FIG. 1, there is shown a horizontal cross-sectional view of a spinal column 10 of a patient in order to illustrate the implanting of an exemplary interbody spacer 12 of the present invention. In particular, there can be seen that the spinal column 10 has a posterior side 14, an anterior side 16 and lateral sides 18, 20 and a interbody spacer can be positioned or implanted in the spinal column 10 intermediate adjacent vertebrae by insertion posteriorly, that is, from the posterior side 14 in the direction of the arrow A, anteriorly, that is, from the anterior side 16 in the direction of the arrow B or translaterally, that is, from each of the lateral sides 18, 20 in the direction respectively, of the arrows C and D. Other features of the spinal column 10 include the pedicle 22 and the spinal cord 24. As can be seen, the overall shape of the cross section of the spinal column 10 basically dictates the kidney shaped outer configuration of the interbody spacer 12.

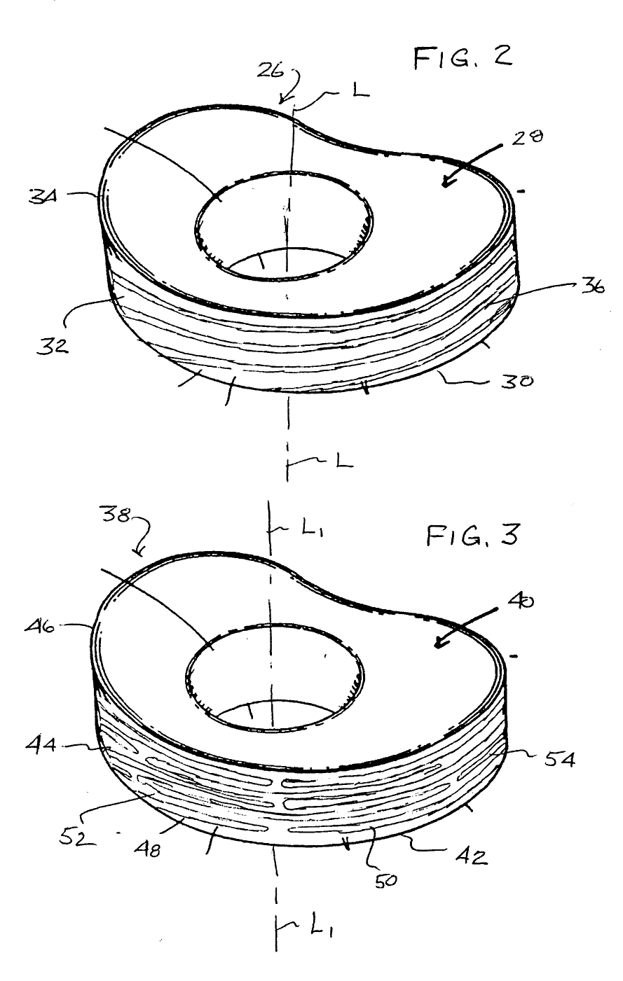

Turning now to FIG. 2, there is a perspective view of an exemplary interbody spacer 26 constructed in accordance with the present invention. As can be seen, the interbody spacer 26 has an upper surface 28 and a lower surface 30 that are generally in parallel planes with respect to each other and an outer peripheral surface 32. In the embodiment shown, the interbody spacer 26 is in the configuration of a kidney shape and which is compatible with the intervertebral space intermediate adjacent vertebrae, referring again to FIG. 1.

In this embodiment, there is a generally tubular body 34 having a longitudinal axis L and having formed therein, a spiral or helical slot 36 formed inwardly along the outer peripheral surface 32 of that tubular body 34. The helical slot 36 may be a continuous slot or a plurality of helical slots and is constructed in accordance with the helical slot shown and described in U.S. Pat. No. 5,488,761 of Leone entitled FLEXIBLE SHAFT AND METHOD FOR MANUFACTURING SAME and the disclosure of that patent is hereby incorporated herein in its entirety by reference.

As described in the Leone patent, the presence of the helical slot creates a flexible, monolithic shaft that can be pre-designed so as to inherently have the desired flexibility for the particular purpose. As stated, during the manufacturing process, the degree of flexibility can be designed into the devices by controlling the pitch and width of the helical slot or slots so that the ultimate device can be custom designed and manufactured with a particular desired flexibility and which is an advantage when designing an interbody spacer that is intended to incorporate a desired flexibility to adjacent vertebrae for a patient's spinal column while still having the advantages of a monolithic construction. With the ability to flex along multiple axes, therefore the flexibility of the interbody spacer can allow the patient to bend at least in the side to side direction as well as the front to back direction.

Turning now to FIG. 3, there is shown an alternative embodiment of an interbody spacer 38 according to the invention and having a different slotted configuration along its longitudinal axis L₁. Again, there is an upper surface 40 and a lower surface 42, generally in parallel planes and an outer peripheral surface 44. In this embodiment, there are pairs of oppositely disposed slots formed in the tubular body 46, that is, there is a pair of oppositely disposed, coplanar slots 48, 50 and a second pair of coplanar slots 52, 54 spaced apart along the longitudinal axis L₁ of the tubular body 46. The oppositely disposed slots are formed in the outer peripheral surface 44 and are directed inwardly and extend up to but not beyond the midpoint of the tubular body 46. The slots are formed as shown and described in copending U.S. patent application Ser. No. ______ of Jaime Martinez, filed Jun. 3, 2005 and the disclosure of that patent application is hereby incorporated herein in its entirety by reference.

Again, due to the shape and the location of the slots in the FIG. 3 embodiment, the interbody spacer 38 allows flexing of the interbody spacer 38 along multiple axes and allows movement of the patient both in the front to back direction as well as the side to side direction. There may be a plurality of such pairs of slots used with the FIG. 3 embodiment, but no less that two pairs of such slots, that are angularly spaced apart such that adjacent pairs of slots are angularly displaced at a preferred angle of about 90 degrees. Again, as with the FIG. 2 embodiment, the use of the pairs of slots allow a multi-axial flexibility to the tubular body 46 and that flexibility can be predetermined or designed into the interbody spacer 38 in the manufacturing process by varying the width of the slots, the spacing between the slots and, the of course, the material used in constructing the interbody spacer 38.

In any event the FIG. 3 embodiment is a monolithic, flexible interbody spacer 38 that can be implanted intermediate two adjacent vertebrae of a spinal column in the same manner as with the FIG. 2 embodiment and provides a means of flexibility between those vertebrae along multiple axes so that the patient is able to bend, at least, in the front to back direction as well as the side to side direction.

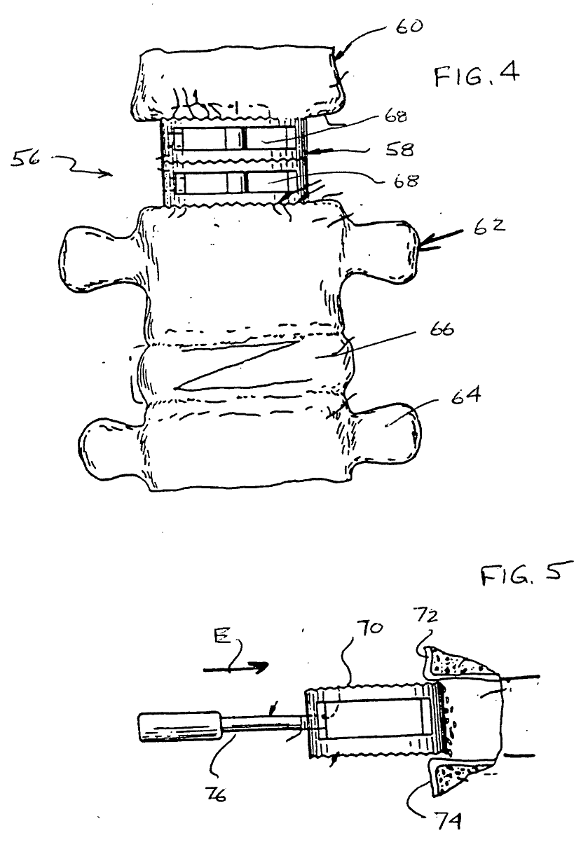

Turning now to FIG. 4, there is shown a front view of a spinal column 56 and illustrating the position of an exemplary interbody spacer 58 of the present invention in its implanted position intermediate adjacent vertebrae 60 and 62. There is also shown a further vertebra 64 and having a healthy disc 66 intermediate the adjacent vertebra 62 and the vertebra 64. The interbody spacer 58 has been implanted between the vertebrae 60, 62 to take up the space formerly occupied by a disc that has been removed and the interbody spacer 58 provides support between the vertebrae 60, 62 while allowing flexibility therebetween along multiple axes with a flexible monolithic device so as to allow the patient to bend in the side to side as well as front to back directions. The presence of slots 68 illustrated in the interbody spacer 58 is basically schematic since the actual slots would be designed to be in the shape and locations of the slots heretofore shown and described with respect to the embodiments of FIGS. 2 and 3.

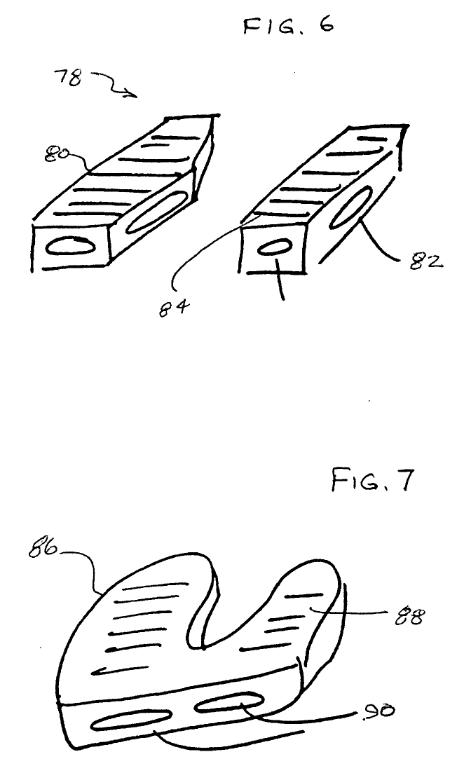

Turning now to FIG. 5, there is shown a side schematic view of an exemplary interbody spacer 70 according to the invention being inserted between two adjacent vertebrae 72, 74 by means of an insertion tool 76 into the intervertebral space 78 therebetween. With the use of the insertion tool 76, the interbody spacer 70 is inserted anteriorly in the direction of the arrow E and is referred to as an anterior lumbar interbody (ALID) device. The insertion tool 76 is affixed to the interbody spacer 70 in order to carry out the insertion and that means of affixation can be by a threaded connection or other means. The interbody device 70 may be any of the interbody spacers of the present invention.

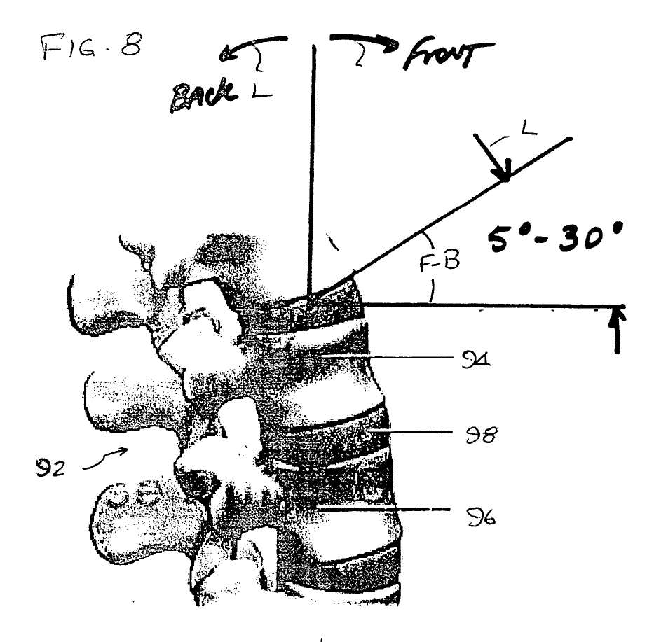

Next, turning to FIG. 6, there is a shown a perspective view of a further interbody spacer 78 according to the invention known as a Posterior Lumbar Interbody Device or a PLID and, as can be seen, the interbody spacer comprises two rectangular devices 80, 82 and are hollow bodies having slots 84, with either the oppositely disposed slots or spiral slot formed therein as previously described with respect to FIGS. 2 and 3.

Turning next to FIG. 7, there is a perspective view of a still further interbody spacer 86 that is a generally U-shaped body 88, e.g., translateral lumbar interbody device (TLID) again with a slot 90 formed therein and, again, the slot 90 may be the spiral slot of FIG. 2 or the pairs of oppositely disposed slots of FIG. 3, the slot 90 being shown in FIG. 7 as a schematic representation of either of those slot configurations.

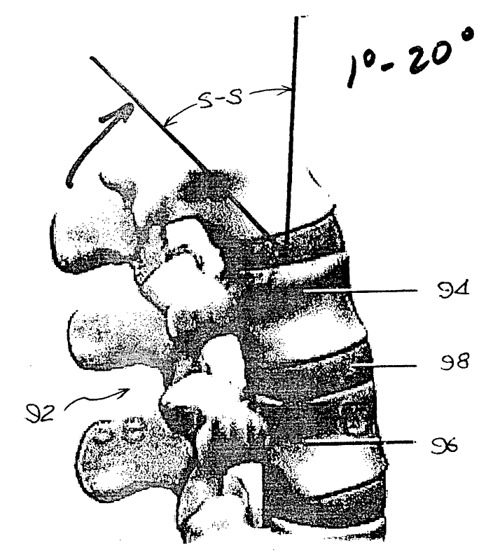

Turning now to FIG. 8, there is shown a side view of a spinal column 92 to illustrate the difference in directional flexibility that is a feature of the present invention. As can be seen, the adjacent vertebrae 94, 96 are separated by a disc 98 and the arrows L indicate the extent of the normal front to back movement of the spinal column 92, that is, the flexion/extension movement of the spinal column 92. As can be seen, the front to back movement of the spinal column 92, shown as angle F-B, is between about greater than 0 to about 30 degrees and with the present interbody spacer, that movement can be designed into the interbody spacer itself in the design and manufacturing stage. In a true biomechanical sense, the movement may be limited to about 10 to about 20 degrees for a single level of the spinal column 92.

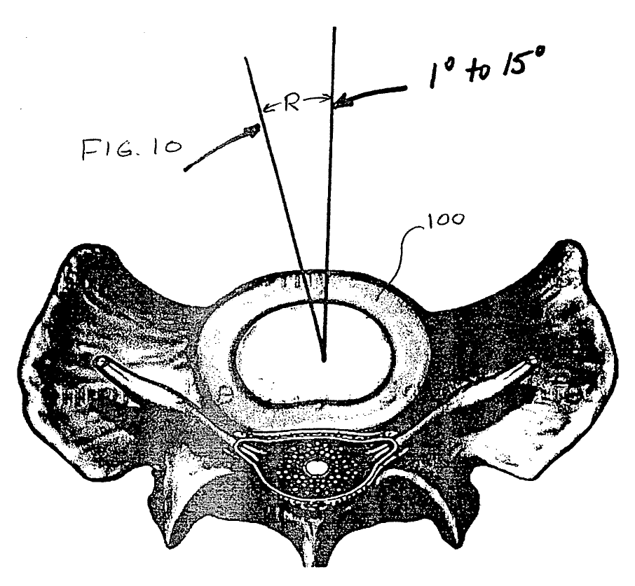

Turning then to FIG. 9, there is shown a side view of the spinal column 92 of FIG. 8 and illustrating the lateral bending or side to side movement of the spinal column 92. As can therefore be seen, the side to side movement is shown in the angle S-S to be from about greater than 0 degrees to about 20 degrees of movement, and, in a true biomechanical sense, that side to side movement may be limited to about greater than 0 degrees to about 10 degrees for a single level of the spinal column 92.

Turning finally to FIG. 10, there is shown a top view of one of the vertebra 100 and illustrating the rotation movement about the axis S and that rotation movement is shown as the angle R to be between greater than about 0 degrees to about 15 degrees and, again the rotational capabilities in a true biomechanical sense may be limited to about greater than about 0 degrees to about 5 degrees of rotational movement for a single level of a spinal column.

As therefore illustrated, the present interbody spacer is capable of being constructed so as to allow the movement in the front to back, side to side and rotational directions to suit the normal biomechanical movement of a patient.

Those skilled in the art will readily recognize numerous adaptations and modifications which can be made to the interbody device and method of constructing the same of the present invention which will result in an improved interbody and method, yet all of which will fall within the scope and spirit of the present invention as defined in the following claims. Accordingly, the invention is to be limited only by the following claims and their equivalents.

What is claimed is:

1. An interbody spacer comprising:
a monolithic device having first and second ends for anchoring to facing surfaces of adjacent vertebrae of a patient; and
said monolithic device further having a flexible region disposed between said first and second ends so as to enable a relative degree of movement of said adjacent vertebrae along multiple axes and allowing differing degrees of flexing in differing directions.

2. The interbody spacer of claim 1 wherein the flexible region in the monolithic device is comprised of a body having at least one slot formed therein.

3. The interbody spacer of claim 2 wherein the body is tubular and has a spiral slot formed therein.

4. The interbody spacer of claim 3 wherein the degree of movement between adjacent vertebrae in the front to back direction is in the range from about greater than 0 to about 20 degrees.

5. The interbody spacer of claim 3 wherein the degree of movement between adjacent vertebrae in the side to side direction is in the range from about greater than 0 to about 30 degrees.

6. The interbody spacer of claim 3 wherein the degree of rotational movement between adjacent vertebrae is in the range from about greater than 0 to about 15 degrees.

7. The interbody spacer of claim 2 wherein the body is tubular and has at least two pairs of oppositely disposed slots displaced along a longitudinal axis of the tubular body and where adjacent pairs of oppositely disposed slots are angularly displaced with respect to each other.

8. The interbody spacer of claim 7 wherein a pair of oppositely slots is displaced about 90 degrees with respect to an adjacent pair of oppositely disposed slots.

9. The interbody spacer of claim 7 wherein the degree of movement between adjacent vertebrae in the side to side direction is in the range from about greater than 0 to about 30 degrees.

10. The interbody spacer of claim 7 wherein the degree of movement between adjacent vertebrae in the front to back direction is in the range from about greater than 0 to about 20 degrees.

11. The interbody spacer of claim 1 wherein the multiple axes allow a patient to move at least along the directions of side to side and front to back.

12. An interbody spacer for insertion intermediate adjacent vertebrae of the spine of a patient, said interbody spacer comprising a body having an upper surface adapted to engage a vertebrae and a lower surface adapted to engage an adjacent vertebrae, the body having a longitudinal axis and an outer peripheral surface,
the body having formed therein at least one of (a) a first and second pair of oppositely disposed slots, each pair being coplanar and in a plane transverse to the longitudinal axis of the tubular body, the first and second pair of oppositely disposed slots being radially offset an angular amount with respect to each other, or (b) a spiral slot,
whereby motion is allowed between the adjacent vertebrae along multiple axes with differing degrees of flexure movement.

13. The interbody spacer as defined in claim 12 wherein the multiple axes include differing side to side motion of a patient and front to back motion of a patient.

14. The interbody spacer as defined in claim 12 wherein the body is a tubular body having a first and second pair of oppositely disposed slots, each pair being coplanar and in a plane transverse to the longitudinal axis of the tubular body, the first and second pair of oppositely disposed slots being radially offset with respect to each other an angular amount formed in the peripheral surface of the tubular body.

15. The interbody spacer as defined in claim 14 wherein each of the first and second pairs of oppositely disposed slots are formed in the tubular body to extend inwardly toward but not reaching the midpoint of the tubular body to form web sections between the respective pairs of slots.

16. The interbody spacer of claim 12 wherein the predetermined angle of radial displacement between the first and second pairs of oppositely disposed slots is about ninety degrees.

17. The interbody spacer of claim 12 wherein the body is a U-shaped body adapted to be inserted translaterally between adjacent vertebrae.

18. The interbody spacer of claim 12 wherein the body is at least one rectangular body adapted to be inserted posteriorly between adjacent vertebrae.

19. A method of implanting an interbody spacer between adjacent vertebrae of a spinal column, said method comprising the steps of:
providing a monolithic device having first and second ends and a flexible region disposed between said first and second ends;
implanting the monolithic device intermediate adjacent vertebrae of a patient so as to enable a relative degree of movement of adjacent vertebrae along multiple axes and with differing amounts of flexure movement.

20. The method of claim 19 wherein the step of providing a monolithic device comprises providing a U-shaped monolithic device and where the step of implanting the monolithic device comprises inserting the U-shaped device between adjacent vertebrae translaterally.

21. The method of claim 19 wherein the step of providing a monolithic device comprises providing a rectangular monolithic device and where the step of implanting the monolithic device comprises inserting the rectangular device between adjacent vertebrae posteriorly.

22. The method of claim 19 wherein the step of providing a monolithic device comprises providing a monolithic device having a body with at least one slot formed therein.

23. The method of claim 22 wherein the at least one slot comprises a spiral slot formed on the body.

24. The method of claim 22 wherein the at least one slot comprises at least two pairs of oppositely disposed slots displaced along a longitudinal axis of the body and where adjacent pairs of oppositely disposed slots are angularly displaced with respect to each other.
